(19) United States
(12) Patent Application Publication
(10) Pub. No.: US 2021/0290740 A1
(43) Pub. Date: Sep. 23, 2021

(54) NEUROTOXIN B FOR USE IN THE TREATMENT OF SKIN DISEASES

(71) Applicants: Universitetssykehuset Nord-Norge HF, Tromsø (NO); International Health Care Management Sweden AB, Stockholm (SE)

(72) Inventors: Øystein Grimstad, Tromsø (NO); Bjorn Øyvind Kvammen, Trondheim (NO); Carl Swartling, Uppsala (SE)

(21) Appl. No.: 17/261,070

(22) PCT Filed: Jul. 23, 2019
(86) PCT No.: PCT/EP2019/069785

§ 371 Date: Jan. 18, 2021

(30) Foreign Application Priority Data
Jul. 23, 2018 (EP) 18185003.3

(51) Int. Cl. A61K 38/48; A61K 9/00; A61P 17/00; A61P 17/12

(52) U.S. Cl. CPC A61K 38/4893; A61P 17/12; A61P 17/00; A61K 9/0021

(57) ABSTRACT

The present invention relates to Botulinum toxin B for use in treatment of at least one skin disease selected from the group consisting of Hidradenitis Suppurativa, Hailey-Hailey disease and Palmoplantar keratodermas.

The present invention relates to botulinum toxin B for use in treatment of at least one skin disease selected from the group consisting of Hidradenitis Suppurativa, Hailey-Hailey disease and Palmoplantar keratodermas. Further, the present invention relates to a pharmaceutical composition comprising botulinum toxin B and one or more pharmaceutically acceptable excipients for use in treatment of at least one skin disease selected from the group consisting of Hidradenitis Suppurativa, Hailey-Hailey disease and Palmoplantar keratodermas.

### BACKGROUND OF THE INVENTION

Hidradenitis suppurativa or acne inversa (HS) is a chronic skin follicular disease characterized by recurrent, painful, deep-seated, inflamed, rounded nodules usually ending in abscesses and sinus tracts with suppuration and hypertrophic scarring of apocrine gland bearing skin. Further, foul-smelling pus secretion from the affected areas is prevalent. The most common locations of HS are the apocrine gland bearing skin of the axillary, inguinal and anogenital regions. The social life as well as the sexual life of HS patients are often hindered or ruined by the scarring and foul-smelling secretions. Thus, has a severe, negative impact on the quality of life of the patients. The prevalence of HS in Europe is 1% and the onset of the disease is typically in young adulthood. The exact cause of HS remains unknown. The HS patients can be classified into three groups by the Hurley staging system, which is based largely on the presence and extent of cicatrization and sinuses. The stages are known as Hurley stage I, II, and III in which stage III is the most severe (Wollina et al., Indian Dermatol Online J., 2013:4). Treatment of Hidradenitis suppurativa requires constant management and is frequently difficult to manage. Medical treatment may include antibiotics, antibiosis, acitretin, and biologic immunosuppressants such as TNFα inhibitors. Recently botulinum toxin A has also been described in single case studies of patients with HS. Use of light and laser therapy in treatment of HS is also being evaluated. Effects of many of the documented treatments are often limited or absent, and there is little evidence of the efficacy of the different treatments in randomized clinical trials or after long-term follow-up. In severe or persistent cases surgery may be required (Feito-Rodrguez et al., American society for dermatological surgery, 2009:35).

Hailey-Hailey disease (familial benign chronic pemphigus) is an inherited skin condition affecting the skin folds of the groin, armpits, neck and under the breasts. The initial lesion may be a red, scaly area or a fluid filled blister which ruptures easily and becomes macerated or crusted. The affected areas are foul-smelling and itching, which may be a social distress to patients' lives. Furthermore, bacterial, fungal, and viral infections are common in the affected areas. There is no cure for Hailey-Hailey disease. Treating patients for the symptoms does provide patients with relief and reduces the microbial infections, e.g. by soothing compresses, topical corticosteroids and antibiotics. Resistant cases that fail topical treatment have responded well to the following; oral corticosteroids, dapsone, topical tacrolimus, and photodynamic therapy. Dermabrasion, laser treatment and surgical skin grafting can also be used in severe cases (Hailey-Hailey disease leaflets, British Association of Dermatologists, www.bad.org.uk/leaflets, last update dec 2016).

Palmoplantar keratodermas (PPK) are a heterogeneous group of disorders characterized by abnormal thickening of the palms and soles (hyperkeratosis). PPK can be divided into three forms: diffuse, striated, and punctate. The pathogenesis of PPK remains unknown, and the treatment is purely symptomatic. There is no definitive treatment or cure. Treatment modalities consist of topical and systemic therapy including retinoids, corticosteroids, calcipotriol, and keratolytics which may be useful in reducing the thickness of the keratoderma. But the lesions recur when treatment is stopped. Surgical excision has also been tried. Overall, the outcomes of treatment of PPK have been rather disappointing (Lienemann et al., Cutis, 2004:73(5)).

Accordingly, there is an urgent need for treatment of the above described skin diseases Hidradenitis suppurativa, Hailey-Hailey disease and Palmoplantar keratodermas.

### SUMMARY OF THE INVENTION

The present invention provides in a first aspect botulinum toxin B for use in treatment of at least one skin disease selected from the group consisting of Hidradenitis Suppurativa, Hailey-Hailey disease and Palmoplantar keratodermas.

According to one embodiment of the present invention, botulinum toxin B is administered by intradermal injection.

According to one embodiment of the present invention, botulinum toxin B is administered by multiple intradermal injections in an area or areas affected by the skin disease.

According to one embodiment botulinum toxin B administered within a period of 3 month does not exceed 4000 Units (U)/patient.

According to one embodiment botulinum toxin B administered within a period of 3 month does not exceed 2000 1000, 500, 400, 300, 250, 200, 100 or 50 Units (U)/patient.

According to one embodiment, each single injection comprises 0.5-7 units (U) botulinum toxin B.

According to one embodiment, each single intradermal injection comprises 2.5-5 units (U) botulinum toxin B.

In one embodiment, the botulinum toxin B is injected intradermally in a concentration of 50 Units (U)/ml and in a volume of 0.05-0.1 ml/injection.

In one embodiment the multiple intradermal injections are given with a distance of 0.5-2.0 cm between each injection.

In one embodiment the multiple intradermal injections are given with a distance of 1.0-1.5 cm between each injection.

In one embodiment botulinum toxin B is administered at a dose of 0.5-10 Units (U)/cm2 in an area affected by the skin disease.

In one embodiment botulinum toxin B is administered at a dose of 1-5 Units (U)/cm2 in an area affected by the skin disease.

In one embodiment botulinum toxin B is administered at a dose of 2-4 Units (U)/cm2 in an area affected by the skin disease.

In one embodiment botulinum toxin B is administered every 2-6 month.

In one embodiment botulinum toxin B is administered every 3^{rd} month.

In one embodiment botulinum toxin B is administered every 4^{th} month.

According to one embodiment the treatment is maintenance treatment of the skin diseases.

In one embodiment, botulinum toxin B is administered to a patient that also suffers from hyperhidrosis.

The present invention provides in a second aspect a pharmaceutical composition comprising botulinum toxin B and one or more pharmaceutically acceptable excipients for use according to any of the embodiments disclosed above.

The present invention provides in a third aspect a method of treating at least one skin disease selected from the group consisting of Hidradenitis Suppurativa, Hailey-Hailey disease and Palmoplantar keratodermas by administration of botulinum toxin B according to any of the embodiments disclosed above.

### BRIEF DESCRIPTION OF THE DRAWINGS

FIG. 1: Hailey-Hailey disease before treatment, right axilla

FIG. 2: Hailey-Hailey disease after treatment, right axilla

FIG. 3: Hailey-Hailey disease before treatment, lower stomach

FIG. 4: Hailey-Hailey disease after treatment, lower stomach

FIG. 5A: Hailey-Hailey disease before treatment, back

FIG. 5B: Hailey-Hailey disease before treatment, back

FIG. 6: Hailey-Hailey disease after treatment, back

FIG. 7: Palmoplantar Keratoderma before treatment, left foot

FIG. 8: Palmoplantar Keratoderma before treatment, right foot

FIG. 9: Palmoplantar Keratoderma after treatment, left foot

FIG. 10: Palmoplantar Keratoderma after treatment, right foot

### DETAILED DESCRIPTION OF THE INVENTION

Unless specifically defined herein, all technical and scientific terms used have the same meaning as commonly understood by a skilled artisan in the fields of genetics, biochemistry, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail.

Where a numeric limit or range is stated, the endpoints are included. Also, all values and subs ranges within a numerical limit or range are specifically included as if explicitly written out.

The present inventors have surprisingly found that botulinum toxin B has advantageous and desirable characteristics useful in the treatment of the skin diseases Hidradenitis suppurativa, Hailey-Hailey disease and Palmoplantar keratodermas.

Botulinum toxin is a group of neurotoxins produced by e.g. the bacterium Clostridium botulinum, which cause life-threatening neuro-paralysis (Butulism). There are seven distinct serotypes of botulinum toxins, designated A through G, of which A and B are used clinically. Botulinum toxin A is the most studied and most commonly used. Commercially forms of botulinum toxin A (e.g. onabotulinumtoxin A, abobotulinumtoxin A, and incobotulinumtoxin A marketed under the brand names Botox, Dysport and Xeomin, respectively) have a long history of therapeutic and cosmetic uses e.g. as aesthetic medicine and in treatment of focal hyperhidrosis and various muscle spasms. The commercially form of the lesser used botulinum toxin B is rimabotulinumtoxin B (marketed under the brand names Myobloc and NeuroBloc) (Al-Ghamdi et al., Journal of Dermatology and Dermatologic Surgery, 2015:19; Forbat et al., JEADV, 2016).

The seven botulinum toxin serotypes share a common structural organization of one heavy chain and one light chain. These toxins inhibit the release of the neurotransmitter acetylcholine at the neuromuscular junction via a three-stage process: 1) heavy chain mediated neurospecific binding of the toxin, 2) internalization of the toxin by receptor-mediated endocytosis, and 3) ATP and pH dependent translocation of the light chain to the neuronal cytosol where it acts as a zinc-dependent endoprotease cleaving polypeptides (SNARE proteins) essential for neurotransmitter release. Botulinum toxin A binds to and cleaves the 25-kD SNARE protein named SNAP-25 (synaptosome-associated protein of 25 KDa), whereas botulinum toxin B specifically has been demonstrated to binds to and cleaves Vesicle Associated Membrane Protein (VAMP also known as synaptobrevin). VAMP is a component of the protein complex responsible for docking and fusion of the synaptic vesicle to the presynaptic membrane, a necessary step to neurotransmitter release (FDA-approved Labeling of Jul. 31, 2009, 2009). The duration of action or rather inhibition of neurotransmitter release varies among the serotypes of the botulinum toxins based on the half-life of the light chain and the time of the neuron to restore the SNARE proteins. The clinical effects of the botulinum toxins are reversible. Studies suggest that botulinum toxin A has the longest half-life, followed by serotypes CI, B, F, and E. Botulinum toxin A has many different approved clinically uses, whereas the sole approved indication of botulinum toxin B is the treatment of adults with cervical dystonia, in which it is used as a muscle relaxant. The different preparations of botulinum toxins available are not interchangeable, and results with preparations of botulinum toxin cannot be extrapolated to another. It is further believed that botulinum toxin B has more autonomic side-effects than botulinum toxin A. Studies have shown that botulinum toxin B injections for cosmetic applications have a more rapid onset of action and greater area of diffusion at the expense of more painful injections and shorter duration of effects. Botulinum toxin B is only available as a liquid with an acidic pH (5.5-6.5), which explains the increased discomfort associated with its injections.

Therapeutically available botulinum toxin formulations contain variable percentages of inactive toxin that contribute to the overall protein load without contributing to efficacy. For this reason, the potency of all commercially available botulinum toxins are expressed in terms of units of biologic activity. Thus, “units” or just “U” refer to units of potency of a neurotoxin and are mouse lethality units. I.e. one unit of botulinum toxin corresponds to the calculated median intraperitoneal lethal dose (LD50) in mice. However, many factors affect the mouse LD50 bioassay including mouse strain, sex, age, volume and route of injection, time of examination after injection, and delivery vehicle or reconstituting buffer. Moreover, the LD50 units of botulinum toxin products are not standardized across manufacturers. Due to the lack of LD50 bioassay harmonization, the unit potencies of botulinum toxin formulations cannot easily be compared. The units of botulinum toxin B appear to be significantly less effective compared with botulinum toxin A units. (Walker et al., J Clin Aesthet Dermatol, 2014:7(2); Forbat et al., JEADV, 2016; Nigam et al., Indian J Dermatol, 2010:55(1); Scaglione, Toxins (Basel), 20168(3)).

The present inventors have now surprisingly shown that botulinum toxin B injections is effective as treatment of the skin diseases Hidradenitis suppurativa, Hailey-Hailey disease and Palmoplantar keratodermas. Said uses of botulinum toxin B (BoNT-B) are described in further details below.

Thus, a first aspect of the present invention relates to botulinum toxin B for use in treatment of at least one skin disease selected from the group consisting of Hidradenitis Suppurativa, Hailey-Hailey disease and Palmoplantar keratodermas.

In one embodiment according to the present invention, the skin disease is Hidradenitis Suppurativa. In another embodiment according to the present invention, the skin disease is Hailey-Hailey disease. In jet another embodiment according to the present invention, the skin disease is Palmoplantar keratodermas.

In one embodiment according to the present invention, botulinum toxin B is administered by intradermal injections in an area or areas affected by the skin disease.

In one embodiment according to the present invention, botulinum toxin B is administered with multiple injections in the affected skin area. According to one embodiment the multiple injections are given with a distance of 0.1-4.0 cm between each injection, such as given with a distance of 0.3-3.0 cm between each injection.

According to one embodiment the injections are given with 0.5-2.0 cm of distance between each injection, such as given with a distance of 1.0-1.5 cm between each injection.

In one embodiment according to the present invention, the botulinum toxin B administered to one patient within a period of 3 months does not exceed 4000 units (U), which is a maximum dose well known to the skilled person working with botulinum toxin B. Higher doses may increase the risk of systemic side effects.

In a preferred embodiment of the present invention, the botulinum toxin B administered to one patient within a period of 3 months does not exceed 2000 Units (U), such as 1000 Units (U), 500 Units (U), 400 Units (U), 300 Units (U), 250 Units (U), 200 Units (U), 100 Units (U) or 50 Units (U).

In one embodiment of the present invention, botulinum toxin B is administered intradermal with multiple injections in the affected skin areal, each individual injection comprising 0.1-50, such as 0.2-10 units (U) botulinum toxin B.

In one embodiment of the present invention, botulinum toxin B is administered intradermal with multiple injections in the affected skin areal, each individual injection comprising 0.5-7, such as 2.5-5 units (U) botulinum toxin B.

In one embodiment of the present invention, botulinum toxin B is administered intradermal with multiple injections in the affected skin area, wherein the individual injections have a volume of 0.01-1.0 ml and comprise botulinum toxin B in a concentration of 10-500 Units (U)/ml.

In a preferred embodiment of the present invention, botulinum toxin B is administered intradermal with multiple injections in the affected skin area, wherein the individual injections have a volume of 0.05-0.1 ml and comprise botulinum toxin B in a concentration of 50 Units (U)/ml.

Depending on the number of intradermal injection, distance between the intradermal injections and the number of Units (U) per intradermal injection, botulinum toxin B is administered intradermally with multiple injections in the affected skin area, wherein the dose of botulinum toxin B comprises 0.5-10 Units (U)/cm², such as 1-5 Units (U)/cm² or 2-4 Units (U)/cm².

In one embodiment of the present invention, botulinum toxin B is administered intradermally with multiple injections in the affected skin area, wherein the dose of botulinum toxin B comprises 0.5-10 Units (U)/cm², such as 1-5 Units (U)/cm² or 2-4 Units (U)/cm².

Depending on the effect and the duration of the effect of botulinum toxin B treatment in the individual patient, administration of botulinum toxin B is repeated every 2^{nd} to 9^{th} months, such as every 2^{nd} 5^{th}, 6^{th}, 7^{th}, 8^{th}, or 9^{th} month, preferable every 3^{rd} or 4^{th} month.

In one embodiment of the present invention, botulinum toxin B is administered every 2nd-9th months, such as every 2^{nd}-6^{th} months, such as every 2^{nd}-4^{th} months, preferably every 3^{rd}-4^{th} month. Treatment with botulinum toxin B is maintained for as long as the patient require the treatment or management of the skin disease. Especially, for patients suffering from Hailey-Hailey disease the treatment with botulinum toxin B is expected to be a lifelong treatment.

In one embodiment of the present invention, botulinum toxin B is used as maintenance treatment of the skin diseases.

Non-limiting examples of patient groups which can be administered botulinum toxin B for treatment of the skin diseases of the present invention is adults, older people, and children. It has been observed by the present inventors that treatment with botulinum toxin B is especially effective in Hidradenitis Suppurativa and Hailey-Hailey patients if they further are also suffering from hyperhidrosis. I.e. it is speculated that the sweating in these patient is an influencing factor in the pathogenesis of Hidradenitis Suppurativa and Hailey-Hailey; and when the sweating in the treated area is decreased, the symptoms of Hidradenitis Suppurativa and Hailey-Hailey are decreased especially well.

A second aspect of the present invention relates to a pharmaceutical composition comprising botulinum toxin B and one or more pharmaceutically acceptable excipients for use in treatment of at least one skin disease selected from the group consisting of Hidradenitis Suppurativa, Hailey-Hailey disease and Palmoplantar keratodermas.

In one embodiment according to the second aspect of the present invention, the skin disease is Hidradenitis Suppurativa. In another embodiment according to the second aspect, the skin disease is Hailey-Hailey disease. In jet another embodiment according to the second aspect, the skin disease is Palmoplantar keratodermas.

In one embodiment according to the second aspect, the pharmaceutical composition comprising botulinum toxin B is administered by intradermal injections in an area or areas affected by the skin disease.

In one embodiment according to the second aspect, the pharmaceutical composition comprising botulinum toxin B is administered with multiple injections in the affected skin area. According to one embodiment the multiple injections are given with a distance of 0.1-4.0 cm between each injection, such as given with a distance of 0.3-3.0 cm between each injection.

According to one embodiment the injections are given with 0.5-2.0 cm of distance between each injection, such as given with a distance of 1.0-1.5 cm between each injection.

In one embodiment according to the second aspect, the pharmaceutical composition comprising the botulinum toxin B administered to one patient within a period of 3 month does not exceed 4000 units (U), which is a maximum dose well known to the skilled person working with botulinum toxin B. Higher doses may increase the risk of systemic side effects.

In a preferred embodiment, the pharmaceutical composition comprising the botulinum toxin B administered to one patient within a period of 3 months does not exceed 2000 Units (U), such as 1000 Units (U), 500 Units (U), 400 Units (U), 300 Units (U), 250 Units (U), 200 Units (U), 100 Units (U) or 50 Units (U).

In one embodiment of the second aspect, the pharmaceutical composition comprising botulinum toxin B is administered intradermally with multiple injections in the affected skin areal, each individual injection comprising 0.1-50, such as 0.2-10 units (U) botulinum toxin B.

In a preferred embodiment of the second aspect of the invention, the pharmaceutical composition comprising botulinum toxin B is administered intradermally with multiple injections in the affected skin areal, each individual injection comprising 0.5-7, such as 2.5-5 units (U) botulinum toxin B.

In one embodiment of the second aspect, the pharmaceutical composition comprising botulinum toxin B is administered intradermally with multiple injections in the affected skin area, wherein the individual injections have a volume of 0.01-1.0 ml and comprise botulinum toxin B in a concentration of 10-500 Units (U)/ml.

In a preferred embodiment of the second aspect, the pharmaceutical composition comprising botulinum toxin B is administered intradermally with multiple injections in the affected skin area, wherein the individual injections have a volume of 0.05-0.1 ml and comprise botulinum toxin B in a concentration of 50 Units (U)/ml.

Depending on the number of intradermal injection, distance between the intradermal injections and the number of Units (U) per intradermal injection, the pharmaceutical composition comprising botulinum toxin B is administered intradermally with multiple injections in the affected skin area, wherein the dose of botulinum toxin B comprises 0.5-10 Units (U)/cm², such as 1-5 Units (U)/cm² or 2-4 Units (U)/cm².

In one embodiment of the second aspect, the pharmaceutical composition comprising botulinum toxin B is administered intradermaly with multiple injections in the affected skin area, wherein the dose of botulinum toxin B comprises 0.5-10 Units (U)/cm², such as 1-5 Units (U)/cm² or 2-4 Units (U)/cm².

Depending on the effect and the duration of the effect of botulinum toxin B treatment in the individual patient, administration of the pharmaceutical composition comprising botulinum toxin B is repeated every 2^{nd} to 9^{th} months, such as every 2^{nd}, 5^{th}, 6^{th}, 7^{th}, 8^{th}, or 9^{th} month, preferable every 3^{rd} or 4^{th} month.

In one embodiment of the second aspect, the pharmaceutical composition comprising botulinum toxin B is administered every 2nd-9th months, such as every 2^{nd}-6^{th} months, such as every 2^{nd}-4^{th} months, preferably every 3^{rd}-4^{th} month. Treatment with the pharmaceutical composition comprising botulinum toxin B is maintained for as long as the patient require the treatment or management of the skin disease. Especially, for patient suffering from Hailey-Hailey the treatment with botulinum toxin B is expected to be a lifelong treatment.

In one embodiment of the second aspect, the pharmaceutical composition comprising botulinum toxin B is used as maintenance treatment of the skin diseases.

Non-limiting examples of patient groups which can be administered the pharmaceutical composition comprising botulinum toxin B for treatment of the skin diseases of the present invention is adults, older people, and children. It has been observed by the present inventors that treatment with botulinum toxin B is especially effective in Hidradenitis Suppurativa and Hailey-Hailey patients if they further are also suffering from hyperhidrosis. I.e. it is speculated that the sweating in these patient is an influencing factor in the pathogenesis of Hidradenitis Suppurativa and Hailey-Hailey; and when the sweating in the treated area is decreased, the symptoms of Hidradenitis Suppurativa and Hailey-Hailey are decreased especially well.

### Definitions

As used herein “units or (U)” is Type B Units when referring to dosing of botulinum toxin B of the present invention. Said Type B Units are specific for the botulinum toxin B product used in the examples of the present disclosure, i.e. specific for the botulinum toxin B product sold under the trade name Neuroblock. According to the EMEA Summary of Product Characteristics for Neuroblock the Type B Units are not interchangeable with the units used to express the potency of other botulinum toxin preparations (see section 4.4; http://www.ema.europa.eu/docs/en_GB/document_library/EPAR_-_Product_Information/human/000301/WC500026906.pdf).

As used herein, “maintenance treatment”, is a treatment given to help keep the disease to come back after it has disappeared following an initial therapy.

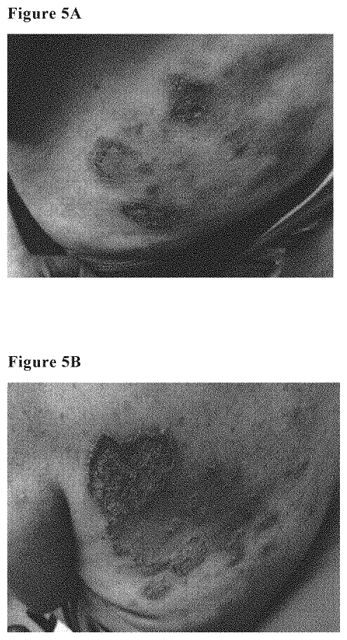

As used herein terms «the affected area» or “an area affected by skin disease” is the area where one can visually observe symptoms of at least one of the diseases selected from the group consisting of Hidradenitis Suppurativa, Hailey-Hailey disease and Palmoplantar keratodermas. In FIG. 5a one can see dark areas of the skin as an example of affected areas. The affected area has any shape and the area can be calculated by applying standard mathematical methods for calculating areas of geometrical shapes, for example by multiplying the length (L) by the width (W) for rectangular shaped affected areas or by applying the formula πr² for circular shaped affected areas, and is expressed in cm².

As used herein a “treatment of the affected area” refers to administration of multiple intradermal injections of botulinum toxin B, where the intradermal injections are given with a set distance between each intradermal injection in the area affected by skin disease.

As used herein the term “area of treatment” refers to the area that receives treatment with botulinum toxin B.

As used herein the term “dose” can be quantified as Units (U) or as “Units (U)/cm²”.

The dose of botulinum toxin B in Units (U) is expressed by the formula:

No. of injections×(U/injection)=Units (U)

The dose of botulinum toxin B in units (U)/cm² is expressed by the formula:

$$\frac{No . of injections \times ( U \text{/} injection )}{Area of treatment in {cm}^{2}} = U \text{/} {cm}^{2}$$

These are examples of calculation of doses for square or rectangular shaped affected areas.

Distance between intradermal injections are 1 cm and dose per intradermal injection is 2.5 Units (U)

For an area of 5×5 intradermal injections:

$$\frac{5 \times 5 \times 2.5 U \text{/} intradermal injection}{4 cm \times 4 cm} = 3.9 U \text{/} cm 2$$

For an area of 10×10 intradermal injections:

$$\frac{10 \times 10 \times 2.5 U \text{/} intradermal injection}{9 cm \times 9 cm} = 3.1 U \text{/} cm 2$$

For an area of 10×10 intradermal injections:

$$\frac{20 \times 10 \times 2.5 U \text{/} intradermal injection}{19 cm \times 19 cm} = 2.9 \text{U} \text{/} cm 2$$

Example of low dose: Distance between intradermal injections are 1.5 cm and dose per intradermal injection is 2.5 Units (U)

For an area of 20×10 intradermal injections:

$$\frac{20 \times 10 \times 2.5 U \text{/} intradermal injection}{28.5 cm \times 13.5 cm} = 1.3 U \text{/} {cm}^{2}$$

Example for high dose: Distance between intradermal injections are 1 cam and dose per intradermal injection is 5 Units (U)

For an area of 5×5 intradermal injections:

$$\frac{5 \times 5 \times 5 U \text{/} intradermal injection}{4 cm \times 4 cm} = 7.8 U \text{/} {cm}^{2}$$

Having generally described this invention, a further understanding can be obtained by reference to certain specific examples, which are provided herein for purposes of illustration only, and are not intended to be limiting unless otherwise specified.

### EXAMPLES

### Example 1: Treatment of Hidradenitis Suppurativa

A randomized double blind placebo controlled study was executed to see if intradermal injection with botulinum toxin B is an effective treatment of Hidradenitis suppurativa.

The study, which was carried out by the University Hospital of North Norway, was performed with triple masking (participant, investigator, outcomes assessor). Participant and investigator/outcomes assessor were blinded. Treatment was given after randomization by a secondary investigator.

Twenty patients (adults of both sexes, minimum 18 years old) with hidradenitis suppurativa were enrolled and randomized to treatment with either botulinum toxin B or placebo (saline) in affected areas.

The inclusion criteria were: Patients with active hidradenitis in the stage I-III according to Hurleys classification. Patients are referred to a dermatology out-patient clinic or patients already in an established treatment program, where there is indication for new or different treatment, or surgical intervention. Patients must have typical affection of the disease of either axillae, groins, and/or perigenital/perianal area.

The exclusion criteria were: Patients in need of emergency medical or surgical treatment of hidradenitis will be excluded until the disease is in a quiet, controlled phase. Pregnant or lactating, as well as patients with neurological disease such as myasthenia gravis or motor neuron disease.

Intervention and recording of data was performed every three months. After three months, all patients received the active substance. After six months, randomization was revealed. Patients with clinical improvement was given the opportunity to continue treatment for additional six months.

### Study Arms

Experimental: Botulinum Toxin B

Botulinum toxin B (Neurobloc®) 50 Units (U)/ml 0.05-0.1 ml/injection intradermally in a grid with 1-1½ cm between every injection in affected areas. A maximum of 4000 U/patient/treatment. Treatment every three months.
Placebo Comparators: Placebo

Saline (NaCl 0.9%) 0.05-0.1 ml/injection intradermally in a grid with 1-1½ cm between every injection in affected areas. Treatment in placebo group (n=10) only at first intervention.

### Outcome

The primary outcome was measured by Dermatological Life Quality Index (DLQI)-scores. DLQI is a measurement of the how much a skin problem affects the life of a patient. It is based on a questionnaire in which the patient evaluates how much their skin influences their everyday life by answering 10 questions. The answers are given a score (0, 1, 2, or 3), which is summarized to a final score (http://www.bad.org.uk/shared/get-file.ashx?id=1653&itemtype=document). The higher the score, the more the quality of life is impaired, see table 1:

TABLE 1
How to interpret meaning of DLQI scores

| DLQI total score | The effect of the skin on patient's life |
|---|---|
| 0-1 | no effect at all on patient's life |
| 2-5 | small effect on patient's life |
| 6-10 | moderate effect on patient's life |
| 11-20 | very large effect on patient's life |
| 21-30 | extremely large effect on patient's life |

As can be seen from tables 2 and 3, improvement after invention with Botulinim toxin B is observed compared to placebo.

TABLE 2
Primary outcome: DLQI after treatment with Botulinim
toxin B; patients treated and examined every 3th month (0 mo.,
3^{rd} mo., 6^{th} mo., 9^{th} mo.)

| | | DLQI | DLQI | DLQI | DLQI |
|---|---|---|---|---|---|
| | | 0 mo., | 3^{rd} mo., | 6^{th} mo., | 9^{th} mo., |
| | | BoNT-B | BoNT-B | BoNT-B | BoNT-B |
| | | inter- | inter- | inter- | inter- |
| patient | affected area | vention | vention | vention | vention |
| 2 | Groin/nates | 22 | 10 | 13 | 11 |
| 4 | Groin/axilla | 12 | 6 | 5 | 7 |
| 6 | Axilla | 11 | 0 | 2 | 0 |
| 8 | Groin | 13 | 6 | 0 | 3 |
| 9 | Axilla | — | — | — | — |
| 12 | Groin/perianal | 16 | 11 | 23 | 14 |
| 13 | Groin/nates | 22 | 17* | 21 | — |
| 16 | Groin | 18 | 17 | 15 | — |
| 17 | Axilla | 19 | 13 | 17 | — |
| 20 | Groin | 22 | 8 | 17 | — |

*patient was having surgery between month 0 and 3^{rd}

TABLE 3
Primary outcome: DLQI after treatment with placebo (saline) and after
the 3th month treatment with Botulinim toxin B; patients treated
and examined every 3th month (0 mo., 3^{rd} mo., 6^{th} mo., 9^{th} mo.)

| | | DLQI | DLQI | DLQI | DLQI |
|---|---|---|---|---|---|
| | | 0 mo., | 3^{rd} mo., | 6^{th} mo., | 9^{th} mo., |
| | | saline | BoNT-B | BoNT-B | BoNT-B |
| | | inter- | inter- | inter- | inter- |
| patient | affected area | vention | vention | vention | vention |
| 1 | Groin/nates | 3 | 5 | 7 | 5 |
| 3 | Groin/nates | 14 | 16 | 6 | 6 |
| 5 | Groin | 12 | 6 | 5 | 7 |
| 7 | Axilla | 5 | 5 | 5 | — |
| 10 | Groin | 17 | 21 | 3 | 0 |
| 11 | Groin | 17 | 17 | 16 | — |
| 14 | Axilla | 2 | 1 | 0 | — |
| 15 | Groin/nates | 11 | 17 | 8 | — |
| 18 | Groin | 21 | 7 | 1 | — |
| 19 | Groin/nates | 2 | 5 | 7 | — |

Further, secondary endpoints were physicians scored measures, hereunder lesion count in the area affected by Hidradenitis Suppurativa.

In tables 4 and 5 the number of lesions in the affected areas is shown. As can be seen from said tables, the number of lesions decreases after treatment with Botulinum toxin B compared to placebo.

TABLE 4
Secondary endpoint: Lesions after treatment with
Botulinim toxin B; patients treated and examined every 3th month
(0 mo., 3^{rd} mo., 6^{th} mo., 9^{th} mo.)

| | | lesions | lesions | lesions | lesions |
|---|---|---|---|---|---|
| | | 0 mo., | 3^{rd} mo., | 6^{th} mo., | 9^{th} mo., |
| | | BoNT-B | BoNT-B | BoNT-B | BoNT-B |
| | | inter- | inter- | inter- | inter- |
| patient | affected area | vention | vention | vention | vention |
| 2 | Groin/nates | 2 | 0 | 0 | 0 |
| 4 | Groin/axilla | 8 | 1 | 1 | 0 |
| 6 | Axilla | 5 | 0 | 1 | — |
| 8 | Groin | 4 | 2 | 0 | 0 |
| 9 | Axilla | 9 | 4 | — | — |
| 12 | Groin/perianal | 5 | 2 | 0 | 0 |
| 13 | Groin/nates | 8 | 2* | 4 | — |
| 16 | Groin | 8 | 6 | 0 | — |
| 17 | Axilla | 3 | 0 | 0 | — |
| 20 | Groin | 5 | 2 | 2 | — |

*patient was having surgery between month 0 and 3^{rd}

TABLE 5
Secondary endpoint: Lesions after treatment with placebo
(saline) and after the 3th month treatment with Botulinim
toxin B; patients treated and examined every 3th month (0 mo., 3^{rd}
mo., 6^{th} mo., 9^{th} mo.)

| | | lesions | lesions | lesions | lesions |
|---|---|---|---|---|---|
| | | 0 mo., | 3^{rd} mo., | 6^{th} mo., | 9^{th} mo., |
| | | saline | BoNT-B | BoNT-B | BoNT-B |
| | | inter- | inter- | inter- | inter- |
| patient | affected area | vention | vention | vention | vention |
| 1 | Groin/nates | 4 | 6 | 5 | 5 |
| 3 | Groin/nates | 6 | 4 | 0 | 0 |
| 5 | Groin | 6 | 9 | 0 | 2 |
| 7 | Axilla | 3 | 3 | 2 | — |
| 10 | Groin | 4 | 4 | 1 | 0 |
| 11 | Groin | 5 | 2 | 2 | — |
| 14 | Axilla | 4 | 1 | 1 | — |
| 15 | Groin/nates | 10 | 2 | 6 | — |
| 18 | Groin | 6 | 5 | 0 | — |
| 19 | Groin/nates | 7 | 1 | 2 | — |

### Example 2: Treatment of Hailey-Hailey Disease

One patient (male adult) with Hailey-Hailey disease was treated with botulinum toxin B in affected areas: axilla, groin, lower stomach, and back.

Before treatment with botulinum toxin B was initiated the patient received systemic therapy with Sandimmun and locally with steroids and needed bandage.

The patient has now for the last 3 years been and is still being treated every third months with botulinum toxin B (Neurobloc®) 50 Units (U)/ml 0.05-0.1 ml/injection intradermally in a grid with 1-1½ cm between every injection in affected areas.

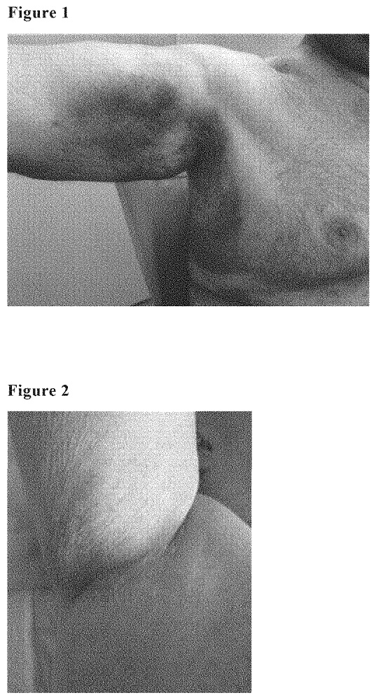

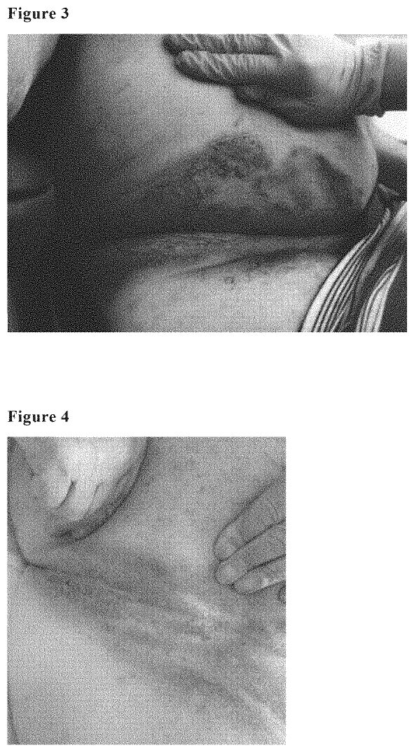

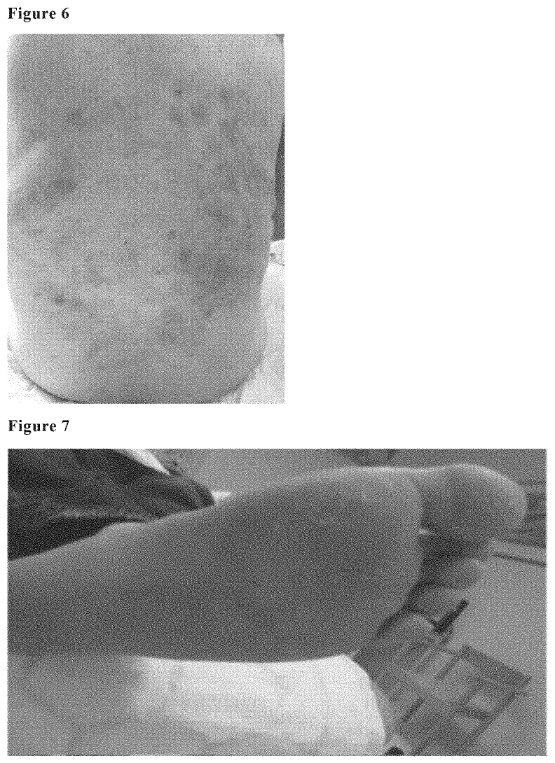

As can be seen from FIGS. 1-6, improvement after intervention with botulinim toxin B is observed in the patient. Further, the patient now does not need treatment with steroids or bandage, and the Sandimmun therapy is now halved.

### Example 3: Treatment of Palmoplantar Keratodermas

Two patients (male adult, aged 32 years and 41 years) with hereditary keratoderma has been treated with botulinum toxin B in affected areas: soles, palms.

For patient #1 (aged 41 years): Before treatment with botulinum toxin B, topical emollients and other topical treatment was without any obvious effect on his hyperkeratosis, primarily affecting the soles of the feet.

For patient #2 (aged 32 years): Systemic treatment with acitretin, topical steroids, topical calcineurin antagonists and emollients has been given without any obvious effect on his hyperkeratosis affecting both palms and soles of the feet.

Patient #1 has now for the 30 months received treatment every third to six months with botulinum toxin B (Neurobloc®) 50 Units (U)/ml 0.05-0.1 ml/injection intradermally in a grid with 1-1½ cm between every injection in affected areas after nerve block anesthesia. Total dose for treating both feet has been in the range of 550-650 IU.

Patient #2 has since 2018 received treatment every four months with Botulinum toxin B (Neurobloc®) 50 Units (U)/ml 0.05-0.1 ml/injection intradermally in a grid with 1-1½ cm between every injection in affected areas in hands after nerve block anesthesia.

Photo documentation showing status prior to treatment with botulinum toxin B only for one of the two patients (Patient #1).

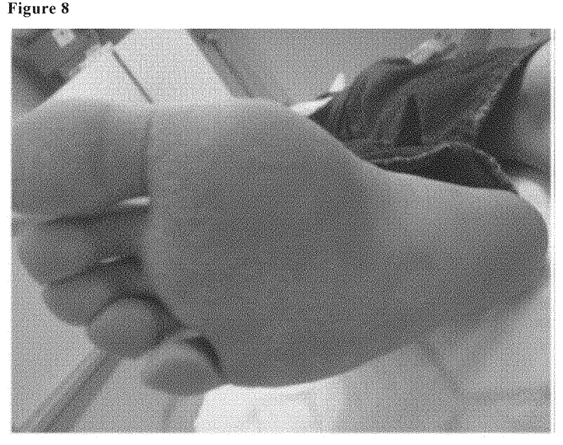

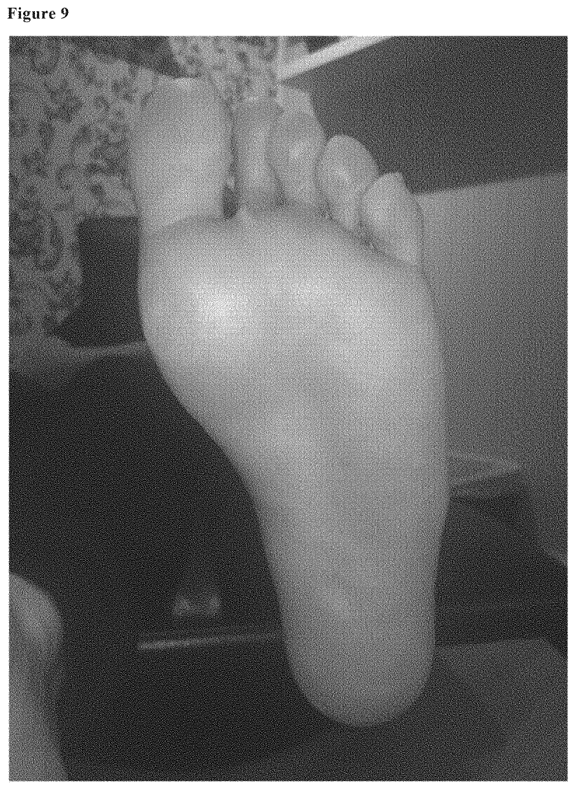

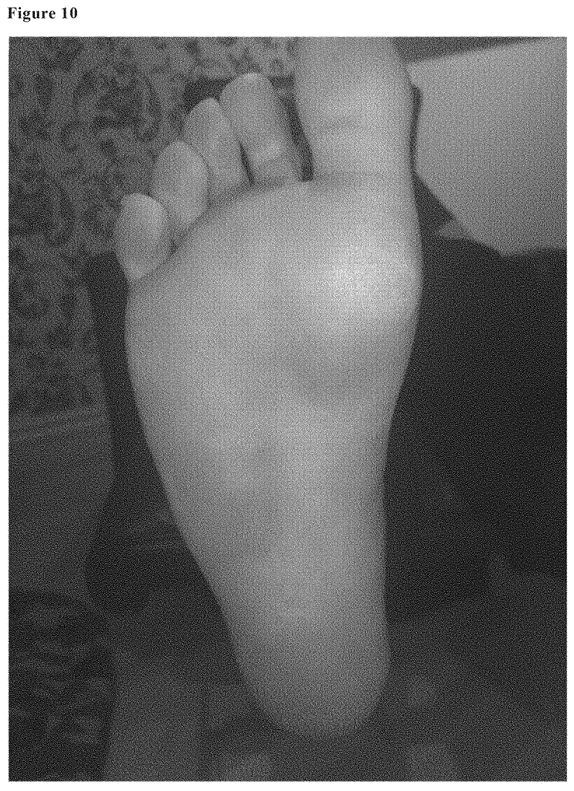

FIGS. 7-10 shows improvement after intervention with Botulinim toxin B observed in this patient.

Both patients now receive Botulinum toxin as monotherapy for their keratoderma disorders on a regular basis.

1. A method of treating at least one skin disease, comprising
administering Botulinum toxin B to a patient in need of such treatment herein the skin disease is selected from the group consisting of Hidradenitis Suppurativa and Palmoplantar Keratoderma,
or
administering Botulinum toxin B to a patient in need of such treatment herein the skin disease is Hailey-Hailey disease, and wherein when the skin disease is Hailey-Hailey disease the botulinum toxin B administered with a period of 3 moths does not exceed 4000 Units (U)/patient.

2. The method according to claim 1, wherein botulinum toxin B is administered by intradermal injection.

3. The method according to claim 2, wherein botulinum toxin B is administered by multiple intradermal injections in an area or areas affected by the skin disease.

4. The method according to claim 1, wherein botulinum toxin B administered to treat of Hidradenitis Suppurativa or Palmoplantar Keratoderma and administering is within a period of 3 months does not exceed 4000 Units (U)/patient.

5. The method according to claim 1, wherein botulinum toxin B administered within a period of 3 month does not exceed 2000, 1000, 500, 400, 300, 250, 200, 100 or 50 Units (U)/patient.

6. The method according to claim 3, wherein each single intradermal injection comprises 0.5-7 units (U) botulinum toxin B.

7. The method according to claim 1, wherein the botulinum toxin B is injected intradermally in a concentration of 50 Units (U)/ml and in a volume of 0.05-0.1 ml/injection.

8. The method according to claim 3, wherein the multiple intradermal injections are given with a distance of 0.5-2.0 cm between each injection.

9. (canceled)

10. The method according to claim 1, wherein the botulinum toxin B is administered at a dose of 1-5 Units (U)/cm² 1 in an area affected by the skin disease.

11. The method according to claim 1, wherein the botulinum toxin B is administered at a dose of 2-4 Units (U)/in an area affected by the skin disease.

12. The method according to claim 1, wherein botulinum toxin B is administered every 2nd-6th month.

13. The method according to claim 1, wherein the treatment is maintenance treatment.

14. The method of claim 1, wherein the patient further suffers from hyperhidrosis.

15. (canceled)
